(19) United States
(12) Patent Application Publication
(10) Pub. No.: US 2019/0388008 A1
(43) Pub. Date: Dec. 26, 2019

(54) SYSTEM AND METHOD FOR RECOGNIZING GAIT

(71) Applicant: National Tsing Hua University, Hsinchu City (TW)

(72) Inventor: Yun-Ju Lee, Hsinchu City (TW)

(73) Assignee: National Tsing Hua University, Hsinchu City (TW)

(21) Appl. No.: 16/150,237

(22) Filed: Oct. 2, 2018

(30) Foreign Application Priority Data
Jun. 21, 2018 (TW) 107121267

(51) Int. Cl. A61B 5/103; G06K 9/00; A61B 5/117

(52) U.S. Cl. CPC A61B 5/1038; A61B 5/112; A61B 5/117; G06K 9/00348

(57) ABSTRACT

The disclosure provides a system and a method for recognizing a gait. The method includes: bearing a first foot of a user by a bearing surface of a sensing device; sensing a gait applied by the first foot to the bearing surface by a sensor of the sensing device; and recognizing the user based on the gait by a processing device.

### CROSS-REFERENCE TO RELATED APPLICATION

This application claims the priority benefit of Taiwan application serial no. 107121267, filed on Jun. 21, 2018. The entirety of the above-mentioned patent application is hereby incorporated by reference herein and made a part of this specification.

### BACKGROUND

### Technical Field

The disclosure relates to a system and a method for biometric recognition, and in particular, to a system and a method for recognizing a gait that is applicable to verification of identity and tracking of a walking trajectory.

### Description of Related Art

Along with the development of biometric recognition techniques based on human body features (e.g., the fingerprint, the iris, the face, etc.), there are more and more applications of techniques related to biometric recognition to the user verification function of terminal devices due to features of biometric information such as uniqueness and inimitability. Currently, in the biometric recognition techniques applied to the user verification function of most terminal devices, user biometric information storage and matching verification are performed through the terminal devices themselves, and there is still a significant risk that the devices may be cracked.

Specifically, since the foregoing biometric recognition methods all involve static recognition, they may have the following disadvantages: (1) recognition may fail due to shading of the human body feature (e.g., the fingerprint, the iris, the face, etc.) to be recognized; (2) the human body feature can be imitated by a high-resolution photograph, which reduces security of the methods; and (3) the foregoing biometric recognition methods all require capturing images of the human body feature by an image-capturing device in advance then performing comparison through image processing. So, the costs of implementation are higher and relevant privacy issues may arise.

Accordingly, for people skilled in the art, how to design a biometric recognition technique that solves the foregoing technical problems is indeed an important issue.

### SUMMARY

In view of the above, the disclosure provides a system and a method for recognizing a gait that can detect a gait of the user and verify the user accordingly, and further solve the foregoing and other technical problems.

The disclosure provides a system for recognizing a gait, including a sensing device and a processing device. The sensing device includes a bearing surface and a sensor. The bearing surface is configured to bear a first foot of a user. The sensor is configured to sense a stepping situation applied by the first foot to the bearing surface. The processing device is coupled to the sensor and converts the stepping situation into a gait of the first foot and recognizes the user based on the gait.

In an embodiment of the disclosure, in a process of stepping where the first foot steps on the bearing surface, the sensor collects a plurality of vibrations consecutively applied by the first foot to the bearing surface, and the stepping situation is characterized by the vibrations.

In an embodiment of the disclosure, the processing device calculates a plurality of center of pressure corresponding to the vibrations based on the vibrations and characterizes a motion trajectory formed of the points of center of pressure as the gait.

In an embodiment of the disclosure, the sensor includes an accelerometer, a gyroscope sensor, a pressure sensor, or an inertial sensor.

In an embodiment of the disclosure, the processing device compares the gait to a predetermined gait, wherein if the gait matches the predetermined gait, the processing device determines that the user is an eligible user, and if not, the processing device determines that the user is an ineligible user.

In an embodiment of the disclosure, the system further includes a plurality of the sensing devices. The sensors of the sensing devices are coupled to the processing device, and the bearing surfaces of the sensing devices coordinately form a specific bearing surface. The sensors of the sensing devices coordinately detect a plurality of specific stepping situations consecutively applied by the first foot and a second foot of the user to the specific bearing surface.

In an embodiment of the disclosure, each of the specific stepping situations is characterized by a motion trajectory, and the processing device connects the motion trajectory corresponding to each of the specific stepping situations to form an integrated motion trajectory and recognizes an activity mode of the user based on the integrated motion trajectory.

In an embodiment of the disclosure, the processing device compares the integrated motion trajectory to a predetermined activity trajectory, wherein if the integrated motion trajectory matches the predetermined activity trajectory, the processing device determines that the activity mode of the user is normal, and if not, the processing device determines that the activity mode of the user is abnormal.

In an embodiment of the disclosure, the motion trajectory corresponding to each of the specific stepping situations is formed by connecting a plurality of center of pressure points generated by the first foot or the second foot on the specific bearing surface.

In an embodiment of the disclosure, the processing device is integrated in the sensing device.

The disclosure provides a method for recognizing a gait including the following steps. A bearing surface of a sensing device bears a first foot of a user. A sensor of the sensing device senses a stepping situation applied by the first foot to the bearing surface. A processing device converts the stepping situation into a gait of the first foot and recognizes the user based on the gait.

In an embodiment of the disclosure, the step of sensing, by the sensor of the sensing device, the stepping situation applied by the first foot to the bearing surface includes the following step. In a process of stepping where the first foot steps on the bearing surface, the sensor collects a plurality of vibrations consecutively applied by the first foot to the bearing surface, wherein the stepping situation is characterized by the vibrations.

In an embodiment of the disclosure, the step of representing, by the processing device, the stepping situation as the vibrations includes the following step. The processing device calculates a plurality of center of pressure points corresponding to the vibrations based on the vibrations and represents the gait as a motion trajectory formed of the center of pressure points.

In an embodiment of the disclosure, the step of recognizing, by the processing device, the user based on the gait includes the following step. The processing device compares the gait to a predetermined gait, wherein if the gait matches the predetermined gait, the processing device determines that the user is an eligible user, and if not, the processing device determines that the user is an ineligible user.

In an embodiment of the disclosure, the method further includes the following step. A plurality of the sensing devices coordinately detects a plurality of specific stepping situations consecutively applied by the first foot and a second foot of the user to a specific bearing surface, wherein the specific bearing surface is coordinately formed by the bearing surfaces of the sensing devices.

In an embodiment of the disclosure, each of the specific stepping situations is characterized by a motion trajectory, and the method further includes the following step. The processing device connects the motion trajectory corresponding to each of the specific stepping situations to form an integrated motion trajectory and recognizes an activity mode of the user based on the integrated motion trajectory.

In an embodiment of the disclosure, the step of recognizing the activity mode of the user based on the integrated motion trajectory includes the following step. The processing device compares the integrated motion trajectory to a predetermined activity trajectory, wherein if the integrated motion trajectory matches the predetermined activity trajectory, the processing device determines that the activity mode of the user is normal, and if not, the processing device determines that the activity mode of the user is abnormal.

In an embodiment of the disclosure, the motion trajectory corresponding to each of the specific stepping situations is formed by connecting a plurality of center of pressure points generated by the first foot or the second foot on the specific bearing surface.

In light of the above, the disclosure provides a system and a method for recognizing a gait that can detect the gait of the user and verify the user accordingly. Since the gait is a dynamic biometric feature, it is not affected by shading and cannot be imitated by a photograph. Moreover, since it is not required to introduce an image-capturing device in the implemented mechanism, it does not trigger the psychological burden associated with the user's impression of being monitored.

To provide a further understanding of the aforementioned and other features and advantages of the disclosure, exemplary embodiments, together with the reference drawings, are described in detail below.

### BRIEF DESCRIPTION OF THE DRAWINGS

FIG. 1 is a schematic diagram illustrating a system for recognizing a gait according to an embodiment of the disclosure.

FIG. 2 is a schematic diagram illustrating a system for recognizing a gait according to an embodiment of the disclosure.

FIG. 3 is a flowchart illustrating a method for recognizing a gait according to an embodiment of the disclosure.

### DESCRIPTION OF THE EMBODIMENTS

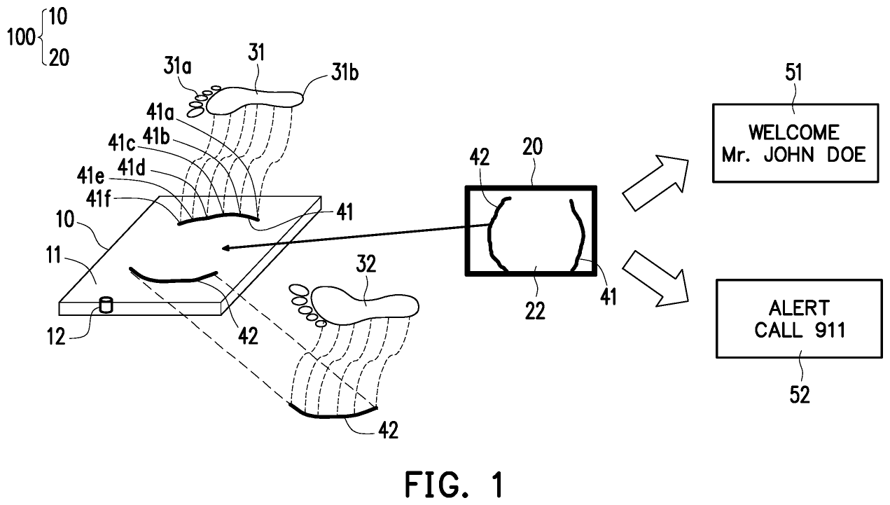

Referring to FIG. 1, FIG. 1 is a schematic diagram illustrating a system for recognizing a gait according to an embodiment of the disclosure. In the present embodiment, a system 100 for recognizing a gait includes a sensing device 10 and a processing device 20. The sensing device 100 includes a bearing surface 11 and a sensor 12. In FIG. 1, the appearance of the sensing device 10 may be implemented as a rectangular plate-shaped object. However, in other embodiments, the sensing device 10 may also be formed of corresponding materials (e.g., acrylic, glass, metal, etc.) and have an appearance (e.g., various geometric shapes) according to the requirements of a designer and is not limited to the example shown in FIG. 1.

In different embodiments, the sensing device 10 may be placed at various locations that require verification of a user. For example, it may be placed in front of an entrance gate that requires verification to pass to serve as an access control system. However, the disclosure is not limited thereto.

In FIG. 1, the bearing surface 11 is, for example, the top surface of the sensing device 10 and may be configured to allow a user A to step on with a foot 31 (e.g., the right foot) and/or a foot 32 (e.g., the left foot) to correspondingly bear the foot 31 and/or the foot 32. It is noted that although FIG. 1 illustrates the scenario of stepping with the feet 31 and 32, in other embodiments, the user A may also step on the bearing surface 11 while wearing shoes, socks, or other similar accessories.

The sensor 12 may be disposed beside the sensing device 10, below the sensing device 10, or may be disposed at any specific position in the sensing device 10 according to the requirements of the designer and is not limited to the example shown in FIG. 1. Moreover, in different embodiments, the sensor 12 may be implemented as one of an accelerometer, a gyroscope sensor, a pressure sensor, and an inertial sensor or a combination of these devices. However, the implementation of the disclosure is not limited thereto.

In the present embodiment, the sensor 12 may be configured to sense a stepping situation applied by the foot 31 and/or the foot 32 to the bearing surface 12. Taking the foot 31 as an example, in the process of stepping of the foot 31 on the bearing surface 11, the sensor 12 collects a plurality of vibrations (which may consist of the values sensed by the accelerometer, the gyroscope sensor, the pressure sensor, or the inertial sensor, for example, but is not limited thereto) consecutively applied by the foot 31 to the bearing surface 11, and the aforementioned stepping situation may be characterized by the vibrations. In an embodiment, the process of stepping of the foot 31 on the bearing surface 11 may include the entire consecutive action from the moment a heel 31b of the foot 31 touches the bearing surface 11 to the moment a foot tip 31a of the foot 31 leaves the bearing surface 11 (which corresponds to the common stepping situation of a human being when walking forward), but the disclosure is not limited thereto. In other embodiments, the process of stepping of the foot 31 on the bearing surface 11 may also be adjusted to other forms according to the requirements of the designer and may include, for example, the entire consecutive action from the moment the foot tip 31a of the foot 31 touches the bearing surface 11 to the moment the heel 31b of the foot 31 leaves the bearing surface 11 (which corresponds to the common stepping situation of a human being when walking backward).

After the sensor 12 collects the vibrations generated in the process of stepping of the foot 31 on the bearing surface 11, the vibrations may be correspondingly provided to the processing device 20. Next, the processing device 20 may convert the stepping situation into a gait of the foot 31 and recognize the user A based on the gait.

In some embodiments, the processing device 20 may be a general-purpose processor, a specific-purpose processor, a conventional processor, a digital signal processor, a plurality of microprocessors, one or more microprocessors combined with digital signal processor cores, a controller, a microcontroller, an application specific integrated circuit (ASIC), a field programmable gate array (FPGA), an integrated circuit of any other type, a state machine, a processor based on an advanced RISC machine (ARM), or a similar device integrated or built in the sensing device 10.

In other embodiments, the processing device 20 may be a mobile phone, a smartphone, a personal computer (PC), a notebook PC, a netbook PC, a tablet PC, or a television connected to the sensing device 10 in a wired/wireless manner, but is not limited thereto.

As an example, in FIG. 1, the processing device 20 is a tablet PC externally connected to the sensing device 10. The tablet PC may include a display 22 for displaying the required information. However, persons of ordinary skill in the art shall understand that the example is not meant to limit the implementation of the disclosure.

In an embodiment, the processing device 20 may calculate center of pressure points 41a, 41b, 41c, 41d, 41e, 41f corresponding to the vibrations and characterizing the gait (which is, for example, the gait of the right foot of the user A) as a motion trajectory 41 formed of the center of pressure points 41a to 41f. Specifically, each of the center of pressure points 41a to 41f is, for example, a central position of the stepped area of the foot 31 on the bearing surface 11 at one time point in the process of stepping of the foot 31. In that case, the motion trajectory 41 may be regarded as a trajectory formed of the central positions of the stepped areas of each of the time points in the process of stepping of the foot 31.

Similarly, the user A may step on the bearing surface 11 with the foot 32 to generate a motion trajectory 42 (which is, for example, a gait of the left foot of the user A), and details of the operation shall be not repeatedly described here. In an embodiment, the motion trajectories 41 and 42 may be further displayed on the display 22 for the user A to confirm.

Since the walking habit and the foot shape differ among different users, the gaits (e.g., the motion trajectories 41 and 42) of different users formed on the bearing surface 11 also differ from person to person and have uniqueness. Therefore, in the disclosure, whether the user A is an eligible user may be recognized based on the gait input by the user A to the bearing surface 11 through the feet 31 and 32.

In an embodiment, the processing device 20 may compare the gait to a predetermined gait. If the gait (e.g., the motion trajectories 41 and 42) matches the predetermined gait, the user A can be determined to be an eligible user. If not, the user A is determined to be an ineligible user.

Specifically, the predetermined gait is, for example, a gait that is previously input to the access control system by an eligible user of the access control system through a method similar to that described above and is used as the basis for verifying a user. In other words, if the user A is an eligible user of the access control system, a manager of the access control system may have the user A perform the operation of inputting the gait to the sensing device 10 as described above in advance in the setting stage and use the input gait as the predetermined gait and store it in a corresponding gait database to be used as the basis for verifying the user A in the future.

If the user A is an eligible user in the first place, the predetermined gait previously input by him/her must match the gait represented by the motion trajectories 41 and 42. Correspondingly, after receiving the gait represented by the motion trajectories 41 and 42, the processing device 20 can verify that the user A is an eligible user and can then perform operations such as granting access, opening the door, turning on a light, unlocking, or another similar operation. Moreover, a welcome message 51 may be displayed on the display 22, but the disclosure is not limited thereto.

Conversely, if the user A is not an eligible user, no corresponding predetermined gait will be found in the gait database of the access control system. Therefore, the processing device 20 determines that the gait represented by the motion trajectories 41 and 42 does not correspond to any predetermined gait in the gait database. In that case, the processing device 20 determines that the user A is an ineligible user and further takes precautions such as issuing an alarm (e.g., displaying a warning message 52 on the display 22), notifying the manager, calling the police, or another similar precaution, but the disclosure is not limited thereto.

According to the description above, the system for recognizing a gait provided in the disclosure can detect the gait of the user and verify the user accordingly. Since the gait is a dynamic biometric feature, it is not affected by shading and cannot be imitated by a photograph. Moreover, since it is not required to introduce an image-capturing device in the implemented mechanism, it does not trigger the psychological burdens associated with the user's impression of being monitored. Furthermore, the system for recognizing a gait of the disclosure can perform recognition without an image processing algorithm. Therefore, the costs of implementation are lower and more financially acceptable. In addition, since the user performs the input of the gait with the two feet, even if the two hands are holding objects, the user can still easily input the gait, which delivers greater convenience.

In other embodiments, in addition to verifying whether the user is an eligible user based on the description above, the system for recognizing a gait of the disclosure can further integrate a plurality of sensing devices to coordinately detect an integrated motion trajectory formed of the motion trajectories corresponding to a plurality of gaits of the user and can determine whether the activity of the user is normal or abnormal based on the integrated motion trajectory, which will be detailed below.

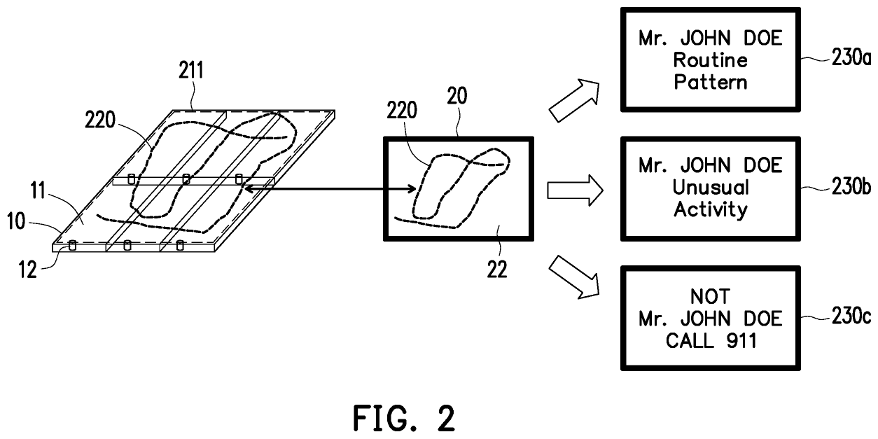

Referring to FIG. 2, FIG. 2 is a schematic diagram illustrating a system for recognizing a gait according to an embodiment of the disclosure. In the present embodiment, a system 200 for recognizing a gait may be distributed, for example, in a specific location (e.g., a factory installed with a plurality of machines) for monitoring whether the activity mode of certain users in the specific location is normal.

As shown in FIG. 2, the system 200 for recognizing a gait includes a plurality of the sensing devices 10 and the processing device 20. Bearing surfaces 11 of the sensing devices 10 in FIG. 2 may coordinately form a specific bearing surface 211.

In FIG. 2, when the user A walks on the specific bearing surface 211 with his/her feet 31 and 32, sensors 12 of the sensing devices 10 can coordinately detect a plurality of specific stepping situations consecutively applied by the user A to the specific bearing surface 211.

Similar to the embodiment of FIG. 1, the specific stepping situations may be characterized by motion trajectories. In other words, each of the specific stepping situations may correspond to one single process of stepping applied by the user A on the specific bearing surface 211 with the foot 31 (or the foot 32). In the process of stepping, each of the sensors 12 collects a plurality of vibrations consecutively applied by the foot 31 (or the foot 32) to the specific bearing surface 211, and the vibrations may characterize one of the specific stepping situations above.

After the sensors 12 collect the vibrations generated in the process of the specific stepping situations of the foot 31 on the specific bearing surface 211, the vibrations may be correspondingly provided to the processing device 20. Next, the processing device 20 may convert the plurality of specific stepping situations into a plurality of gaits corresponding to the user A, and the gaits may correspond to the motion trajectories 41 and 42 as shown in FIG. 1. In other words, each of the gaits may be formed by connecting a plurality of center of pressure points generated by the foot 31 (or the foot 32) on the specific bearing surface 211.

Afterwards, the processing device 20 may connect the gaits (i.e., the motion trajectories) corresponding to the plurality of specific stepping situations to form an integrated motion trajectory 220 and recognize the activity mode of the user A based on the integrated motion trajectory 220.

It is noted that the integrated motion trajectory 220 shown in FIG. 2 is only an example and is not meant to limit the implementation of the disclosure. In other embodiments, since the user A may step on the specific bearing surface 211 alternately with the feet 31 and 32, the formed integrated motion trajectory 220 may be in the form of moving forward in an S shape, but the disclosure is not limited thereto.

In an embodiment, the processing device 20 may compare the integrated motion trajectory 220 to a predetermined activity trajectory. If the integrated motion trajectory 220 matches the predetermined activity trajectory, the activity mode of the user A can be determined to be normal, i.e., the user A currently performs a usual routine. If not, the activity mode of the user A is determined to be abnormal, i.e., the user A currently performs an unusual routine.

The predetermined activity trajectory is, for example, an integrated motion trajectory that is previously input to a related activity management system by the eligible user A of the specific location through a method similar to that described above and is used as the basis for determining whether the activity mode of the user A is normal.

For example, if the routine activity mode of the eligible user A of the specific location is sequentially operating machines X, Y, Z distributed in the specific location, the predetermined activity trajectory previously stored by the user A in the activity management system will be in the form of sequentially passing the machines X, Y, Z.

In that case, if the integrated motion trajectory 220 generated by the user A matches the predetermined activity trajectory (i.e., the form of sequentially passing the machines X, Y, Z), the processing device 20 can determine that the activity mode of the user A is normal (since it is consistent with the routine activity mode of the user A). Correspondingly, the display 22 may display a normal message 230a to let a related manager know that the activity mode of the user A is normal.

However, in other embodiments, if the integrated motion trajectory 220 generated by the user A is in the form of sequentially passing the machines Z, X, Y, the processing device 20 can determine that the integrated motion trajectory 220 does not match the predetermined activity trajectory (i.e., the form of sequentially passing the machines X, Y, Z) and further determine that the activity mode of the user A is abnormal (since it is inconsistent with the routine activity mode of the user A). Correspondingly, the display 22 may display a prompt message 230b to let the related manager know that the activity mode of the eligible user A is abnormal.

In addition, as shown in FIG. 1, if the user A is not an eligible user of the specific location, the display 22 may display a warning message 230c to let the related manager know that the user A is not an eligible user.

It is noted that although the plurality of the sensing devices 10 coordinately detects the activity mode of the user in FIG. 2, in other embodiments, one single sensing device having a larger bearing surface may also be adopted to implement the technical means described above. By disposing a plurality of sensors in such a sensing device, detection precision can be enhanced, and more diversified implementations can be achieved.

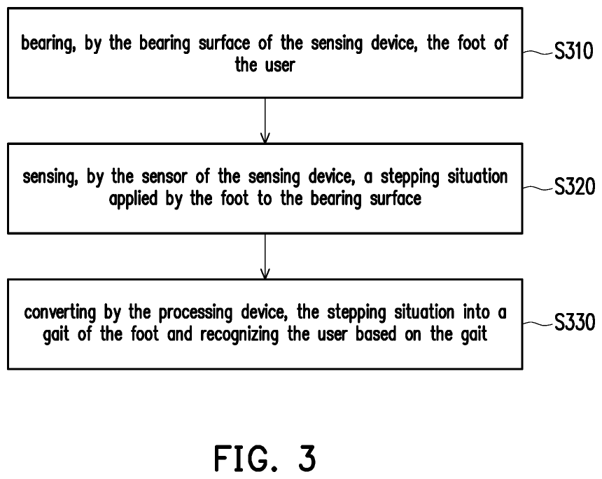

Referring to FIG. 3, FIG. 3 is a flowchart illustrating a method for recognizing a gait according to an embodiment of the disclosure. The method shown in FIG. 3 may be implemented by the system 100 for recognizing a gait of FIG. 1. In the description below, the steps of the method will be described with reference to the components of FIG. 1.

In step S310, the bearing surface 11 of the sensing device 10 bears the foot 31 of the user. In step S320, the sensor 12 of the sensing device 10 senses a stepping situation applied by the foot 31 to the bearing surface 11. In step S330, the processing device 20 converts the stepping situation into a gait of the foot 31 and recognizes the user based on the gait. Reference may be made to the relevant descriptions of FIG. 1 for details of the steps above, which shall not be repeatedly described here.

In other embodiments, after the user is recognized as an eligible user, it may be further recognized whether the activity mode of the user is normal based on the scenario shown in FIG. 2, and various messages shown in FIG. 2 may be correspondingly output according to the result of recognition.

In summary of the above, the system and the method for recognizing a gait provided in the embodiments of the disclosure can detect the gait of the user and accordingly verify whether the user is an eligible user and further achieve at least the following effects: (1) the system and the method are not affected by shading; (2) the gait cannot be imitated by a photograph; (3) the system and the method do not trigger the psychological burdens associated with the user's impression of being monitored; (4) the costs in implementation are lower and more financially acceptable; and (5) even if the two hands are holding objects, the user can still easily input the gait, which delivers greater convenience.

In addition, the system and method for recognizing a gait of the embodiments of the disclosure can further detect the activity mode of the user in a specific location through a large-area specific bearing surface provided by a plurality of sensing devices and accordingly determine whether the activity mode of the user is normal (e.g., being consistent with the routine activity mode of the user) and correspondingly provide relevant prompt messages for the reference of the manager. Thereby, the effect of monitoring the identity/activity mode of the user can be achieved without monitoring of an image-capturing device.

Although the disclosure is disclosed as the embodiments above, the embodiments are not meant to limit the disclosure. Any person skilled in the art may make slight modifications and variations without departing from the spirit and scope of the disclosure. Therefore, the protection scope of the disclosure shall be defined by the claims attached below.

What is claimed is:

1. A system for recognizing a gait, comprising:
a sensing device comprising:
a bearing surface configured to bear a first foot of a user; and
a sensor configured to sense a stepping situation applied by the first foot to the bearing surface; and
a processing device, coupled to the sensor, converting the stepping situation into a gait of the first foot and recognizing the user based on the gait.

2. The system according to claim 1, wherein in a process of stepping where the first foot steps on the bearing surface, the sensor collects a plurality of vibrations consecutively applied by the first foot to the bearing surface, and the stepping situation is characterized by the vibrations.

3. The system according to claim 2, wherein the processing device calculates a plurality of center of pressure points corresponding to the vibrations based on the vibrations and characterizes a motion trajectory formed of the center of pressure points as the gait.

4. The system according to claim 1, wherein the sensor comprises an accelerometer, a gyroscope sensor, a pressure sensor, or an inertial sensor.

5. The system according to claim 1, wherein the processing device compares the gait to a predetermined gait, wherein if the gait matches the predetermined gait, the processing device determines that the user is an eligible user, and if not, the processing device determines that the user is an ineligible user.

6. The system according to claim 1, further comprising a plurality of the sensing devices, wherein the sensors of the sensing devices are coupled to the processing device, and the bearing surfaces of the sensing devices coordinately form a specific bearing surface, wherein the sensors of the sensing devices coordinately detect a plurality of specific stepping situations consecutively applied by the first foot and a second foot of the user to the specific bearing surface.

7. The system according to claim 6, wherein each of the specific stepping situations is characterized by a motion trajectory, and the processing device connects the motion trajectory corresponding to each of the specific stepping situations to form an integrated motion trajectory and recognizes an activity mode of the user based on the integrated motion trajectory.

8. The system according to claim 7, wherein the processing device compares the integrated motion trajectory to a predetermined activity trajectory, wherein if the integrated motion trajectory matches the predetermined activity trajectory, the processing device determines that the activity mode of the user is normal, and if not, the processing device determines that the activity mode of the user is abnormal.

9. The system according to claim 7, wherein the motion trajectory corresponding to each of the specific stepping situations is formed by connecting a plurality of center of pressure points generated by the first foot or the second foot on the specific bearing surface.

10. The system according to claim 1, wherein the processing device is integrated in the sensing device.

11. A method for recognizing a gait, comprising:
bearing, by a bearing surface of a sensing device, a first foot of a user;
sensing, by a sensor of the sensing device, a stepping situation applied by the first foot to the bearing surface; and
converting, by a processing device, the stepping situation into a gait of the first foot and recognizing the user based on the gait.

12. The method according to claim 11, wherein the step of sensing, by the sensor of the sensing device, the stepping situation applied by the first foot to the bearing surface comprises:
in a process of stepping where the first foot steps on the bearing surface, collecting, by the sensor, a plurality of vibrations consecutively applied by the first foot to the bearing surface, wherein the stepping situation is characterized by the vibrations.

13. The method according to claim 12, wherein the step of converting, by the processing device, the stepping situation into the gait of the first foot comprises:
calculating, by the processing device, a plurality of center of pressure points corresponding to the vibrations based on the vibrations and characterizing a motion trajectory formed of the center of pressure points as the gait.

14. The method according to claim 11, wherein the step of recognizing, by the processing device, the user based on the gait comprises:
comparing, by the processing device, the gait to a predetermined gait, wherein if the gait matches the predetermined gait, the processing device determines that the user is an eligible user, and if not, the processing device determines that the user is an ineligible user.

15. The method according to claim 11, further comprising:
coordinately detecting, by a plurality of the sensing devices, a plurality of specific stepping situations consecutively applied by the first foot and a second foot of the user to a specific bearing surface, wherein the specific bearing surface is coordinately formed by the bearing surfaces of the sensing devices.

16. The method according to claim 15, wherein each of the specific stepping situations is characterized by a motion trajectory, and the method further comprises:
connecting, by the processing device, the motion trajectory corresponding to each of the specific stepping situations to form an integrated motion trajectory and recognizing an activity mode of the user based on the integrated motion trajectory.

17. The method according to claim 16, wherein the step of recognizing the activity mode of the user based on the integrated motion trajectory comprises:
comparing, by the processing device, the integrated motion trajectory to a predetermined activity trajectory, wherein if the integrated motion trajectory matches the predetermined activity trajectory, the processing device determines that the activity mode of the user is normal, and if not, the processing device determines that the activity mode of the user is abnormal.

18. The method according to claim 17, wherein the motion trajectory corresponding to each of the specific stepping situations is formed by connecting a plurality of center of pressure points generated by the first foot or the second foot on the specific bearing surface.
